(19) United States
(12) Patent Application Publication
(10) Pub. No.: US 2026/0041335 A1
(43) Pub. Date: Feb. 12, 2026

(54) ETHOD FOR MULTI-LEVEL TONAL ADAPTIVE SCANNING IN AN EXTENDED RANGE OF SOUND FREQUENCIES

(71) Applicant: HEARING REHABILITATION CENTER "MELFON", LTD., Moscow (RU)

(72) Inventors: Valeriy Stepanovich SIROTKIN, g Moskva (RU); Vladimir Vladimirovich KHANYKOV, g Moskva (RU); Sergey Mikhailovich PASYUTA, g. Moskva (RU)

(73) Assignee: HEARING REHABILITATION CENTER "MELFON", LTD., Moscow (RU)

(21) Appl. No.: 19/293,685

(22) Filed: Aug. 7, 2025

(30) Foreign Application Priority Data
Aug. 7, 2024 (RU) 2024122645

(51) Int. Cl. A61B 5/12; H04R 1/10

(52) U.S. Cl. CPC A61B 5/123; H04R 1/10

(57) ABSTRACT

The present invention relates to medicine, namely to the section of preventive medicine, and can be used in screening audiometry for diagnostic purposes during mass preventive examinations of the population. The patient is consistently presented with sound signals in the frequency range: 125-250-500-750-1000-2000-3000-4000-6000-8000-10000-12000-16000 Hz at three tone levels of 20, 30 and 45 dB. In this case, they start with a 20 dB tone signal. If the patient replied “There is a signal” at all frequencies at a signal level of 20 dB, then no further tests are performed and the hearing condition is assessed as excellent. If the patient responds “No signal” at at least one of the frequencies at a signal level of 20 dB, the next frequency pass is triggered at a signal level of 30 dB. And if the patient replied “There is a signal” at all frequencies at a signal level of 30 dB, then the condition is assessed as a potential risk of hearing loss. If the patient responds “No signal” at at least one of the frequencies at a signal level of 30 dB, the next frequency pass is started at a signal level of 45 dB and the condition is assessed as the first degree of hearing loss. At the same time, if the patient responds “No signal” at at least one of the frequencies at a signal level of 45 dB, then the condition is assessed as the second degree of hearing loss. The method allows for an express hearing assessment in a short time period during mass preventive examinations of the population.

### FIELD OF TECHNOLOGY

The present invention relates to medicine, namely to the section of preventive medicine, and can be used in screening audiometry for diagnostic purposes during mass preventive examinations of the population.

### BACKGROUND

To date, tonal hearing screening is almost the only way to obtain “at least some” instrumental assessment of auditory perception in systematically conducted mass examinations in schools. Successful mastering of educational material largely determines not only the successful future of a student, but also the level of future scientific and technological development of the society in which today's students will work. Numerous studies have proven that even a slight hearing impairment significantly reduces the ability to assimilate educational material. WHO has developed the criterion of “educationally Significant Hearing Loss”-ESHL “Educationally Significant Hearing Loss” (AAA, 2011; ASHA, 1997).

The ESHL level is considered to be hearing loss, which interferes with student academic performance (WHO, 2014). This may include permanent sensorineural, conductive, and mixed hearing loss, as well as transient conductive loss. However, the severity of hearing loss that ESHL represents is not always clearly defined. According to the World Health Organization (2014), disabling hearing loss in children is the average threshold of hearing in the best ear at frequencies of 0.5, 1, 2, 4 kHz, which is >30 dB PS.”

In order to reduce labor costs, all existing methods use a narrowed range of the studied “speech” frequencies: 500, 1000, 2000, 4000 Hz, although it is already a well-known fact that high speech frequencies over 6000 Hz have a great influence on speech intelligibility.

The use of manually operated audiometers leads to significant time costs, which leads to the need to use only one volume level value of the scanning test signal. The prognostic effectiveness of the method is sharply reduced and the burden on the healthcare system is greatly increased due to the large number of unconfirmed referrals for additional examination.

From the prior art, a method of two-stage examination of hearing organs in preschool children. At the first stage, the tympanometry method is used, at the second stage, the otoacoustic emission method is used at the distortion product frequency. The examination begins with the use of the tympanometry method at a probing tone frequency of 226 Hz with a pressure change rate from +200 to −400 daPa/s. According to the tympanograms obtained, which have a peak dependence of static compliance on changes in positive or negative air pressure in the external auditory canal, the pressure in daPa/c is estimated, at which the peak of compliance is recorded. If the pressure in the tympanic cavity and the external auditory canal equalizes, and the pressure at which the peak of compliance is recorded ranges from +50 to −100 daPa/c, the function of the middle car is assessed as normal. Otoacoustic emission examination is continued. If a pathology of the middle car is detected according to the results of tympanometry, the child is referred for treatment of the pathology of the middle car. Then, after 3 weeks, repeated tympanometry is performed and the examination is continued using the otoacoustic emission method at the frequency of the distortion product. The registration criterion is the ratio of the emission power to the background noise power of +6 dB in 70% of the frequency bands. If pathology is detected based on the results of otoacoustic emission at the frequency of the distortion product, the child is sent to a sign language center for examination. The method makes it possible to increase the reliability and objectivity of monitoring hearing aid pathology in preschool children (RU 2759485 C1 Nov. 15, 2021).

From the prior art, there is a known method and hardware and software package for performing diagnostic procedures in terms of performing a pre-medical preliminary classifying multifactorial assessment of the capabilities of a human auditory analyzer during mass preventive examinations of the population. A method is proposed for pre-medical preliminary classifying multifactorial assessment of the capabilities of a human auditory analyzer during mass preventive examinations of the population, performed using a computing device connected to audio playback devices and containing stages in which: using a computing device, a primary test speech sequence (TRP) is formed, which consists of sentences consisting of the first number of words in English. based on the matrix test; generate a competing noise sound for the primary TRP; the primary TRP is reproduced using audio signal reproduction devices made in the form of air and bone sound transmission headphones, while the TRP is reproduced simultaneously with the noise competing sound at the first signal-to-noise ratio using speech simulation based on a deep machine learning model; the user's oral response is received; the user's oral response is automatically analyzed by recognizing the TRP converting it to text format and analyzing the correctness of the answer using a machine learning model; moreover, based on the analysis of the user's oral responses, a dynamic change in the complexity of the assessment is carried out, in which, as a result of each automatic analysis, the number of words in sentences forming the TRP and/or the signal-to-noise ratio of the reproduced signal is changed; the user's auditory analyzer is evaluated based on the responses during playback of the test speech sequence. The invention provides an automated pre-medical preliminary classifying assessment of the possibility of a human auditory analyzer during mass preventive examinations of the population (RU 2765108 C1 Jan. 25, 2022).

In addition, a method is known from the prior art in which screening audiometry is performed by applying in semi-automatic mode a sequence of tone signals of a standard set of frequencies separately into each of the channels of the playback device with recording of the user's response by recognizing tone signals.

reproduction of a test sequence of monophonic signals in an extended frequency range for the construction of a screening audiogram (RU 2743049 C1 Feb. 15, 2021).

The disadvantages of this development is a single test sequence at a certain preset test signal level. All frequencies declared for the survey are scanned sequentially. This is a traditional method of tonal audiometry screening, performed on an audiometer with manual adjustment of the volume level of the test signal. During the examination of a group of patients, the required level of the test signal is set and the entire group is examined at this value. If it is necessary to change the level, the procedure is repeated. At each pass, the patient is tested at only one level of the test signal.

Based on the consideration of the identified problematic issues of existing methodological solutions for the organization of screening examinations of schoolchildren (minors), it is possible to formulate requirements for a promising option for tonal screening of auditory perception:

1. It is necessary to abandon the use of a fixed grid of 4 frequencies. For each patient, it is necessary to determine the upper limit of the perceived tonal signals. It is necessary to identify and correct not only “bad” hearing, but also to take care of identifying and maintaining “excellent” hearing.

2. Multi-level organization of screening. The number of hearing scanning levels performed per headphone installation must be adjusted depending on the objectives of the examination. For example, for conducting examinations in schools, we can recommend a three-level screening with sequential testing of one patient at three levels of test signal intensity, providing an operational possibility of automatic “traffic light” classification of the level of his perception of tonal signals:
“Green level”—no signs of abnormalities were detected;
“Yellow level”—signs of a slight impairment in the perception of speech range tonal signals were detected;
“Red level”—signs of impaired perception of tonal signals have been identified. Repeated in-depth examination is necessary.

3. The maximum time of the actual testing process for one patient (from the moment the “start” button is pressed to the end of the test) should not exceed 1.5 minutes for a three-level scan of a mass school student (assuming no neuropsychiatric behavioral abnormalities).

### SUMMARY

The claimed invention solves a technical problem in terms of ensuring the identification of persons without hearing problems “felt” by themselves and their surroundings, but with a tendency to decrease, especially in the field of high speech frequencies, as well as to identify more severe hearing disorders. In addition, the objective of this solution is the possibility of providing increased predictive reliability and reducing the study time.

Thus, the claimed method allows for early diagnosis of hearing loss when a drop in high frequencies is noticed compared to previous measurements, longitudinal analysis and the ability to detail high-frequency tinnitus.

Technically, the result is to enable rapid hearing assessment in a short period of time during mass preventive examinations of the population, to increase diagnostic accuracy through an expanded frequency range, to carry out early diagnosis of hearing loss when a drop in high frequencies is detected compared to previous measurements, longitudinal analysis and the ability to detail high-frequency tinnitus.

The reduction in examination time is ensured by the fact that the patient is presented with an extended frequency range of three tones per study, so instead of examining all patients one at a time at the same testing level, followed by resetting the level (again the same for all), with headphones reinstalling (plus time for ongoing disinfection), it is proposed to perform the entire range of testing for all the frequencies and levels of the test signal of interest for each patient.

The claimed technical result is achieved through the following techniques.

The patient is presented with sound signals in the frequency range sequentially in automatic mode through air-conduction headphones: 125-250-500-750-1000-2000-3000-4000-6000-8000-10000-12000-16000 Hz at three tone levels of 20, 30 and 45 dB, while starting with a tone signal in 20 dB;
if the patient replied “There is a signal” at all frequencies at a signal level of 20 dB, then no further tests are performed and the hearing condition is assessed as excellent;
If the patient responds “No signal” at at least one of the frequencies at a signal level of 20 dB, the next pass is triggered at a signal level of 30 dB at frequencies that were not heard at a signal level of 20 dB;
if the patient replied “There is a signal” at all the presented frequencies at a signal level of 30 dB, then the condition is assessed as a potential risk of hearing loss.
if the patient responds “No signal” at at least one of the frequencies at a signal level of 30 dB, the next pass is triggered at a signal level of 45 dB at frequencies that were not heard by the patient at a signal level of 30 dB;
if the patient replied “there is a signal” at the required frequencies at a signal level of 45 dB, then the condition is assessed as the first degree of hearing loss;
if the patient responds “No signal” at at least one of the frequencies at a signal level of 45 dB, then the condition is assessed as the second degree of hearing loss.

### A BRIEF DESCRIPTION OF THE DRAWINGS

FIG. 1 illustrates the flowchart of the tonal screening algorithm.

FIG. 2 illustrates the general scheme of the software and hardware complex.

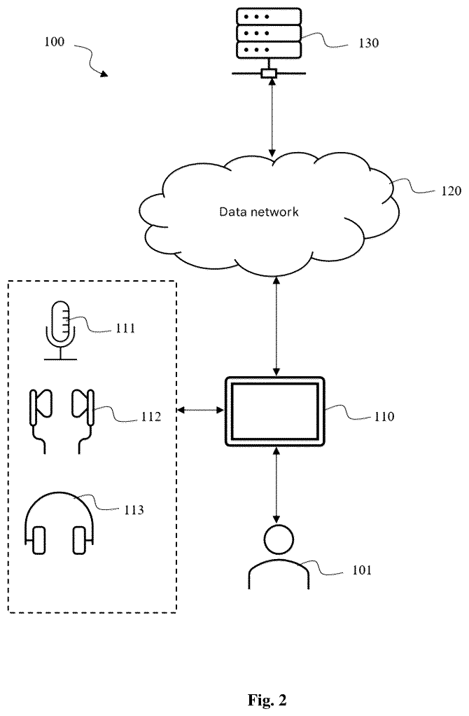

FIG. 3-FIG. 5 illustrate the protocols of tonal screening tests.

### DETAILED DESCRIPTION OF THE INVENTION

To eliminate the existing shortcomings in conducting rapid hearing assessment, in order to organize multi-level screening and examination, for example, in schools, we can recommend three-level screening with sequential testing of one patient at three levels of test signal intensity, providing an operational possibility of automatic classification of the level of his perception of tonal signals. This screening allows you to significantly reduce the time of the actual testing process for one patient with a three-level scan of a mass school student. In addition, the method makes it possible to carry out an early diagnosis of hearing loss when a drop in high frequencies is noticed compared to previous measurements, longitudinal analysis and the ability to identify tinnitus.

TABLE 1
Generally accepted international scale of hearing loss.
Degree of violation Hearing thresholds,

| | | Degree of severity |
|---|---|---|
| Degree of violation | PTA, dB | violations |
| normal | “−10”-25 | normal |
| Grade I hearing loss | 26-40 | light |
| Grade II hearing loss | 41-55 | average |
| Grade III hearing loss | 56-70 | medium |
| Grade IV hearing loss | 71-90 | heavy |
| Deafness | More than 91 | deafness |

Thus, the developed method of rapid hearing assessment is carried out as follows: the patient is presented with sound signals in the frequency range sequentially in automatic mode through air-conduction headphones: 125-250-500-750-1000-2000-3000-4000-6000-8000-10000-12000-16000 Hz at three tone levels of 20, 30 and 45 dB. In this case, they start with a 20 dB tone signal. If the patient replied “There is a signal” at all frequencies at a signal level of 20 dB, then no further tests are performed and the hearing condition is assessed as excellent. If the patient responds “No signal” at at least one of the frequencies at a signal level of 20 dB, the next pass is triggered at a signal level of 30 dB at the frequencies at which the patient replied “no signal” at a signal level of 20 dB. And if the patient replied “There is a signal” at all the presented frequencies at a signal level of 30 dB, then the condition is assessed as a potential risk of hearing loss. If the patient responds “No signal” at at least one of the frequencies at a signal level of 30 dB, the next pass is triggered at a signal level of 45 dB at frequencies that were not heard by the patient at a signal level of 30 DB. If the patient replied “there is a signal” at the required frequencies at a signal level of 45 DB, then the condition is assessed as the first degree of hearing loss. At the same time, if the patient responds “No signal” at at least one of the frequencies at a signal level of 45 dB, then the condition is assessed as the second degree of hearing loss.

At the same time, if the subject does not hear a signal at two consecutive frequencies at frequencies of 4 kHz and above, then the test is not performed at higher frequencies in this ear and it is assumed that there is no signal on them either. This determines the highest frequency that the subject hears.

After conducting such a study on the right and left ear, it is repeated with an increased signal level, for example 40 dB. Only those frequencies for which the subject did not hear at the first level are tested. The limitation for high frequencies is the same as for the first level. Thus, a scheme has been developed to optimize the scanning procedure at all levels, which significantly reduces the total scanning time and allows detecting hearing disorders and tinnitus, which can only be identified using the specified extended frequency range.

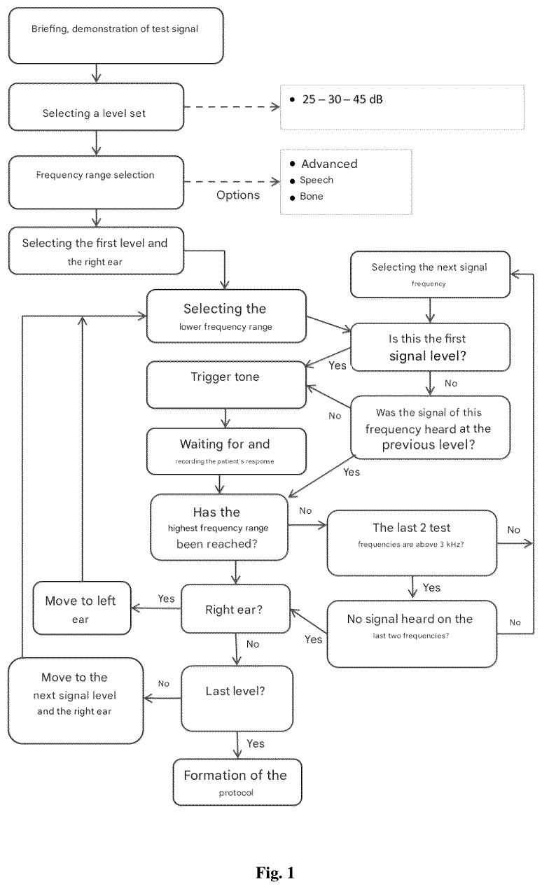

On average, the study takes no more than 2 minutes of rapid diagnosis of both ears. FIG. 1 shows a block diagram of the implementation of the claimed method.

The claimed method can be implemented based on the SURDOSCOPE software and hardware complex (SaHC) (patent RU 2743049 C1, Feb. 15, 2020). FIG. 1 shows the general scheme of the SaHC (100). It includes a computing device (110), which is a tablet, smartphone, laptop or personal computer. The device (110) provides all the necessary functionality to enable interaction with the user (10) to implement the claimed method.

The computing device (110) contains a data input device (111) and audio playback devices (112, 113) connected to it via a data transmission channel.

The speech input device (111) It can be used as an integrated or external microphone. External design of the device (111) It can be connected using any suitable communication principle, for example, USB interface, 2.5/3.5 mm jack, Lightning connector, Bluetooth, radio channel, etc.

The audio playback devices (112, 113) are two types of headphones—air (for example, the Sennheiser hd 400s) and bone conduction (for example, the Aftershock trekz titanium), which provide the possibility of selective two-channel transmission of sound to the user (101) generated by the computing device (110). The headphones (112, 113) are calibrated with verification, for example, using the Tester application, to match the output parameters of the product (volume, frequency) to the audiometric equipment. Calibration parameters must comply with international and/or national standards, for example, GOST R IEC 60645-1-2017. The calibration of the audio playback devices (112, 113) is carried out using an artificial ear, for example, manufactured by B&K.

The computing device (110) can also be connected via a data network (120), for example, the Internet, to a remote server (130), which can store various information, including settings, user data, data update packages, parameters and information for conducting tests, etc.

The computing device (110) provides the generation of all necessary signals, sounds and graphical information for the implementation of the user testing process (101) for the implementation of a multi-level tonal adaptive diagnosis of hearing disorders in an extended range of sound frequencies. This assessment can be carried out using algorithmic execution and activation of tests presented to the user (10).

Along with the above, the claimed method allows, instead of examining all patients one by one at the same test level, followed by resetting the level (again the same for all), with the headphones reinstalled (plus time for ongoing disinfection), it is proposed to perform the entire test package at all frequencies and test signal levels of interest for each patient.

In addition, not only patients with auditory perception disorders are identified, but also patients with a normal level of perception of tonal signals in the extended range up to 16 kHz are identified. It is important not only to treat or correct hearing disorders, but also to ensure the preservation of hearing.

The upper limit of sound perception thresholds is determined for each patient. This indicator is critically important in developing a correction scheme for the detected degree of hearing loss.

### Example 1

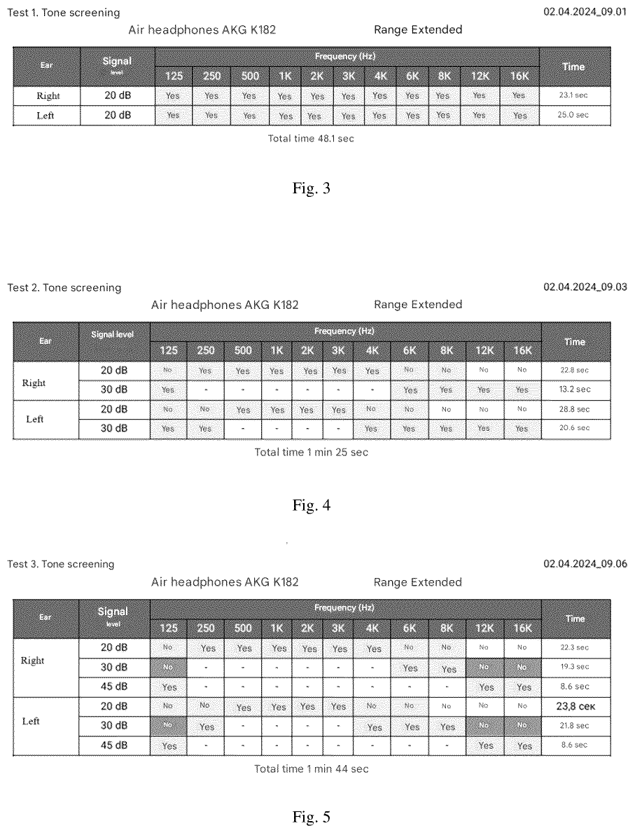

Patient N. does not complain about hearing, tonal screening was performed as part of the medical examination, and the patient was automatically presented with audio signals in the frequency range using headphones: 125-250-500-750-1000-2000-3000-4000-6000-8000-10000-12000-16000 Hz and sequentially The signal is presented at levels initially at a tone level of 20 dB. The patient responded to both ears in turn at all frequency ranges from 125 to 16,000 Hz with a tone level of 20 dB that he hears all the signals. Such results of the rapid test allowed us to conclude that the patient has an excellent hearing level (see FIG. 3).

### Example 2

Patient K. does not complain about hearing, tonal screening was performed as part of the medical examination, and the patient was automatically presented with audio signals in the frequency range using headphones: 125-250-500-750-1000-2000-3000-4000-6000-8000-10000-12000-16000 Hz and consistently present The signal at the levels is initially at a 20 dB tone level. The patient, upon presentation to the right car of sound signals at frequencies of 125, 6000, 8000, 12000 and 16000 Hz with a tone level of 20 dB, indicated that “there is no signal.” Next, the patient is also presented with a 30 dB tone signal in the right ear at frequencies that were not heard at the 20 dB tone signal, namely 125, 6000, 8000, 12000 and 16000 Hz, and the patient replied “there is a signal”. The patient, upon presentation to the left car of sound signals at frequencies of 125, 250, 4000, 6000, 12000 and 16000 Hz with a tone level of 20 dB, indicated that “there is no signal.” Next, the patient is also presented with a 30 dB tone signal in the left car at frequencies that were not heard at the 20 dB tone signal, namely 125, 250, 4000, 6000, 12000 and 16000 Hz, and the patient replied “there is a signal”. Such rapid test results allowed us to conclude that this patient has a potential hearing impairment and a more in-depth examination is required (see FIG. 4).

### Example 3

Patient M. does not complain about hearing, tonal screening was performed as part of the medical examination, and the patient was automatically presented with audio signals in the frequency range using headphones: 125-250-500-750-1000-2000-3000-4000-6000-8000-10000-12000-16000 Hz and consistently present The signal at the levels is initially at a 20 dB tone level. The patient, upon presentation to the right car of sound signals at frequencies of 125, 6000, 8000, 12000 and 16000 Hz with a tone level of 20 dB, indicated that “there is no signal.” Next, the patient is also presented with a 30 dB tone signal in the right car at frequencies that were not heard at a 20 dB tone signal, namely 125, 6000, 8000, 12000 and 16000 Hz, and the patient replied “there is a signal” at frequencies of 6000 and 8000 Hz, and at frequencies of 125, 12000 and the 16,000 patient replied, “There is no signal.” A 45 dB signal was presented to the patient's right ear at frequencies of 125, 12,000, and 16,000 Hz, and a response was received from the patient, “there is a signal.” Upon presentation of sound signals at frequencies of 125-16000 Hz with a tone level of 20 dB to the left car, the patient indicated that there was “no signal” at the frequencies: 125, 250, 6000, 8000, 12000 and 16,000. Next, the patient is also presented with a 30 dB tone signal in the left ear at frequencies that were not heard at a 20 DB tone signal, namely 125, 250, 4000, 6000, 12000 and 16000 Hz, and the patient replied “there is a signal” at frequencies 250, 6000 and 8000, and at frequencies 125, 4000, 12000 and 16000 Hz—“no signal”. A 45 dB signal was presented to the patient's left ear at frequencies of 125, 4000, 12000 and 16000 Hz and a response was received from the patient “there is a signal”. Such results of the rapid test allowed us to conclude about the first degree of hearing loss (see FIG. 5).

In these application materials, a preferred disclosure has been provided for the implementation of the claimed technical solution, which should not be used as limiting other, private embodiments of its implementation, which do not go beyond the scope of the requested scope of legal protection and are obvious to specialists in the relevant field of technology.

1. A method for multi-level tonal adaptive diagnosis of hearing disorders in an extended range of sound frequencies, including presenting the patient with automatic audio signals in the frequency range when the sound signal levels change, characterized in that the range of tested frequencies is used: 125-250-500-750-1000-2000-3000-4000-6000-8000-10000-12000-16000 Hz and sequentially present the signal at the tone levels of 20, 30 and 45 dB, while starting with a tone signal of 20 dB; if the patient replied “There is a signal” at all frequencies at a signal level of 20 dB, then no further tests are performed and the hearing condition is assessed as excellent;
if the patient responds “No signal” at at least one of the frequencies at a signal level of 20 dB, the next pass is triggered at a signal level of 30 dB at frequencies that were not heard at a signal level of 20 dB;
if the patient replied “There is a signal” at all the presented frequencies at a signal level of 30 dB, then the condition is assessed as a potential risk of hearing loss;
if the patient responds “No signal” at at least one of the frequencies at a signal level of 30 dB, the next pass is triggered at a signal level of 45 dB at frequencies that were not heard by the patient at a signal level of 30 dB;
if the patient replied “there is a signal” at the required frequencies at a signal level of 45 dB, then the condition is assessed as the first degree of hearing loss;
if the patient responds “No signal” at at least one of the frequencies at a signal level of 45 dB, then the condition is assessed as the second degree of hearing loss.

2. The method according to claim 1, wherein if the subject does not hear a signal at two consecutive frequencies at frequencies of 4 kHz and above, then the test is not performed at higher frequencies in this ear.

3. The method according to claim 1, wherein the presentation of audio signals to the patient is carried out using headphones.

4. The method according to claim 3, wherein the headphones are air or bone sound transmission.
